(19) United States
(12) Patent Application Publication
(10) Pub. No.: US 2011/0184374 A1
(43) Pub. Date: Jul. 28, 2011

(54) Peristaltic Pump and Cassette

(76) Inventors: Shawn X. Gao, Irvine, CA (US); Brian W. McDonell, Sherman Oaks, CA (US)

(21) Appl. No.: 12/694,572

(22) Filed: Jan. 27, 2010

(51) Int. Cl. A61M 5/142; F04B 43/12

(52) U.S. Cl. 604/500; 417/477.2

(57) ABSTRACT

A cassette for use with a peristaltic pump. The cassette having a body having an exterior. Additionally, the cassette has a sheet attached to the body forming at least one fluid channel. The fluid channel being generally arcuate and projecting outwardly from the exterior of the body. The fluid channel having at least one outlet port for receiving a liquid. Furthermore, the cassette has a liquid trap in fluid communication with and extending at least in part in an upward direction from the at least one outlet port.

### BACKGROUND OF THE INVENTION

The present invention relates generally to peristaltic pumps and more specifically to peristaltic pumps and cassettes used in ophthalmic surgical equipment.

Peristaltic pumps are widely used in medical applications because of their predictable, constant flow properties. Most prior art peristaltic pumps work by compressing or squeezing a length of flexible tubing using a rotating roller head. As the roller head rotates, the rollers pinch off a portion of the tubing and push any fluid trapped in the tubing between the rollers in the direction of rotation.

A peristaltic drain pump used with ophthalmic surgical equipment is often subjected to vacuum at the inlet of the pump. The loading force and the alignment of the drain pump roller head relative to the cassette are critical in order to provide the isolation between the inlet and the outlet of the drain pump. Ideally the pinching action of the pump would completely seal the pump preventing any leakage between the upstream and downstream portions of the pump. However, this is not always the case and small leaks may occur. Such a leak allows fluids (e.g. gas or liquid) to travel back upstream. In the case of current peristaltic pumps, the adverse effect of a leak is worse because these pumps allow gas to be present downstream of a leak. Thus, a faulty seal in current peristaltic pumps enables gas to travel upstream where the gas expands thereby causing problems with the operation of the peristaltic pump.

Accordingly, a need continues to exist for a peristaltic pump that reduces the adverse effects of a leakage between the upstream and downstream portions of the pump.

### SUMMARY OF THE INVENTION

These and other aspects, forms, objects, features, and benefits of the present invention will become apparent from the following detailed drawings and description.

A cassette for use with a peristaltic pump. The cassette having a body having an exterior. Additionally, the cassette has a sheet attached to the body forming at least one fluid channel. The fluid channel being generally arcuate and projecting outwardly from the exterior of the body. The fluid channel having at least one outlet port for receiving a liquid. Furthermore, the cassette has a liquid trap in fluid communication with and extending at least in part in an upward direction from at least one outlet port.

A further cassette for use with a peristaltic pump. The cassette having a body having an exterior. Additionally, the cassette has a sheet attached to the body forming at least one molded fluid channel. The fluid channel being generally arcuate and projecting outwardly from the exterior of the body to define an expansion volume. The fluid channel having at least one outlet port for receiving a liquid. Furthermore, the cassette has a liquid trap in fluid communication with at least one outlet port. The liquid trap defining a volume that is greater than or equal to the expansion volume of the fluid channel.

A method for operating a peristaltic pump having a roller head. The method including providing a surgical cassette. The cassette having a body having an exterior. Additionally, the cassette has a sheet attached to the body forming at least one fluid channel. The fluid channel having an inlet port and an outlet port. Furthermore, the cassette has a liquid trap in fluid communication with and extending at least in part in an upward direction from the outlet port. Additionally, the method includes positioning the cassette with respect to the roller head of the peristaltic pump. Furthermore, the method includes rotating the roller head such that at least one roller contacts the sheet to cause fluid flow through the at least one fluid channel. Also, the method includes trapping liquid with the liquid trap adjacent the outlet port.

### BRIEF DESCRIPTION OF THE DRAWINGS

In the accompanying drawings, which are incorporated in and constitute a part of the specification, embodiments of the invention are illustrated, which, together with a general description of the invention given above, and the detailed description given below, serve to exemplify the embodiments of this invention.

FIG. 1 is a schematic front plan view of a peristaltic pump that may be used with the present invention, with the motor and roller head removed for clarity.

FIG. 2 is a schematic rear plan view of the peristaltic pump of FIG. 1 that may be used with the present invention.

FIG. 3 is a schematic side elevational view of the peristaltic pump of FIG. 1 that may be used with the present invention, with the motor and roller head removed for clarity.

FIG. 4 is a cross-sectional view of the peristaltic pump of FIG. 1 that may be used with the present invention taken at line 4-4 in FIG. 1.

FIG. 5 is a schematical view of a cassette of the peristaltic pump of FIG. 1 positioned within a surgical console that that may be used with the present invention.

### DETAILED DESCRIPTION OF THE INVENTION

The present disclosure relates generally to the field of ophthalmic surgery, and more particularly to peristaltic pumps and cassettes that reduce the adverse effects of a leakage between the upstream and downstream portions of the pump. For the purposes of promoting an understanding of the principles of the invention, reference will now be made to embodiments or examples illustrated in the drawings, and specific language will be used to describe these examples. It will nevertheless be understood that no limitation of the scope of the invention is thereby intended. Any alteration and further modifications in the described embodiments, and any further applications of the principles of the invention as described herein are contemplated as would normally occur to one skilled in the art to which the disclosure relates.

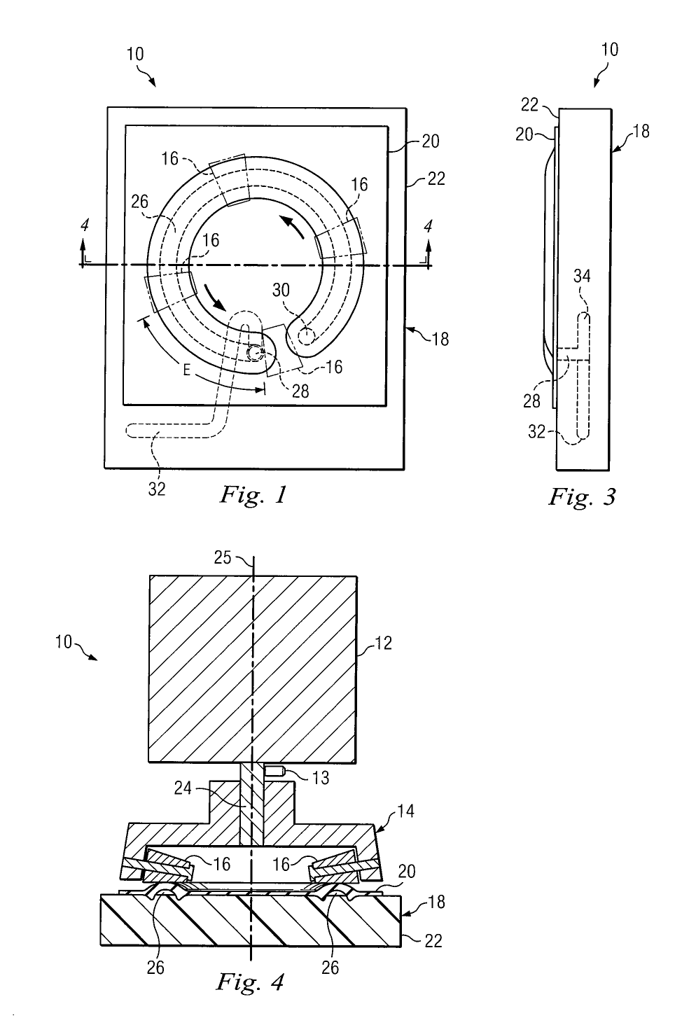

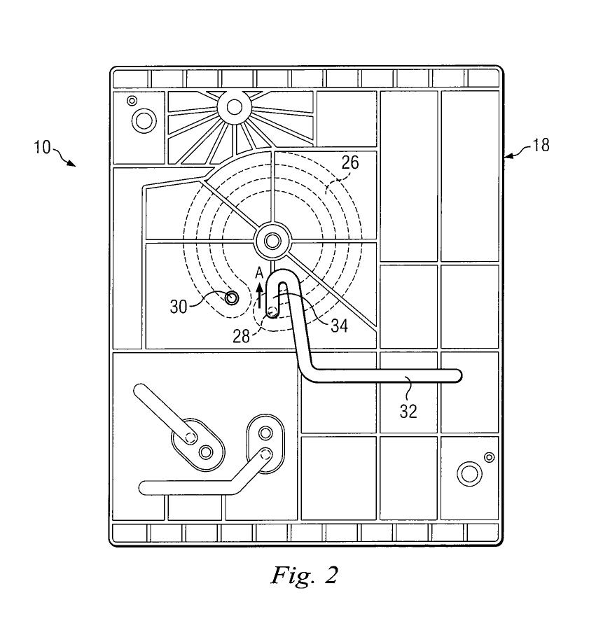

FIGS. 1-4 show various views of an exemplary embodiment of a peristaltic pump. FIG. 1 is a schematic front plan view of a peristaltic pump that may be used with the present invention, with the motor and roller head removed for clarity. FIG. 2 is a schematic rear plan view of the peristaltic pump of FIG. 1 that may be used with the present invention. FIG. 3 is a schematic side elevational view of the peristaltic pump of FIG. 1 that may be used with the present invention, with the motor and roller head removed for clarity. FIG. 4 is a cross-sectional view of the peristaltic pump of FIG. 1 that may be used with the present invention taken at line 4-4 in FIG. 1.

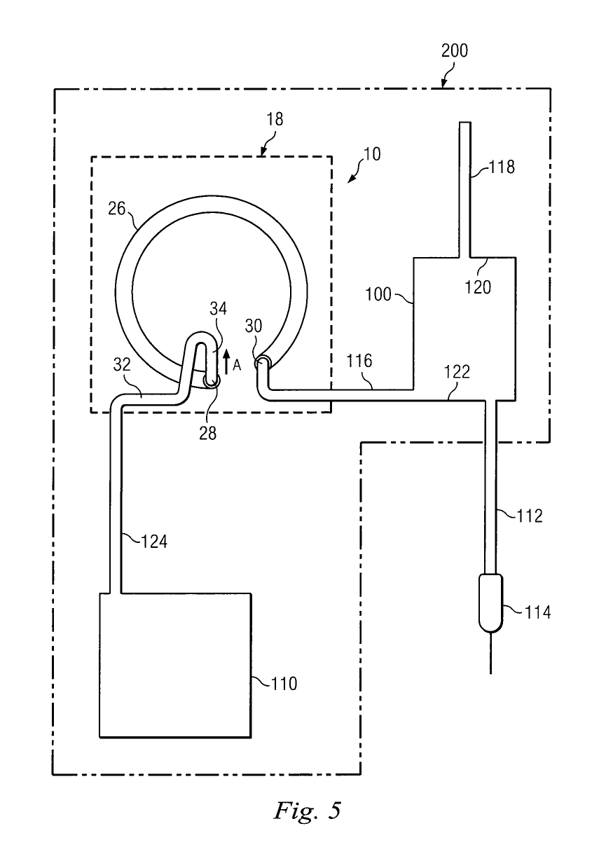

As best seen in FIGS. 1-4, peristaltic pump 10 that may be used with the present invention generally includes pump motor 12, roller head 14, containing one or more rollers 16 and cassette 18 having elastomeric sheet 20 applied to the exterior of relatively rigid body or substrate 22. Pump motor 12 preferably is a stepper or D.C. servo motor. Roller head 14 is attached to shaft 24 of motor 12 so that motor 12 rotates roller head 14 in a plane generally normal to the axis of shaft 24, and the longitudinal axes of rollers 16 are generally radial to the axis of shaft 24. Motor 12 contains encoder 13, which may be located on shaft 24 that provides rotational location information for shaft 24. As the position of rollers 16 is fixed relative to shaft 24, information regarding the rotational information of shaft 24 also locates rollers 16. Pump motor 12, roller head 14, roller 16 and encoder 13 are generally located within surgical console 200, represented schematically only in FIG. 5. Surgical console 200 contains sufficient hardware and software to process signals from encoder 13 and control motor 12. Suitable hardware and software is well-known to those skilled in the art and one suitable console is the INFINITI® Vision System available from Alcon Laboratories, Inc., Fort Worth, Tex.

Sheet 20 contains molded fluid channel 26 that is generally planar, arcuate in shape (within the plane) and having a radius approximating that of rollers 16 about shaft 24. Fluid channel 26 fluidly connects outlet port 28 and inlet port 30. Sheet 20 may be made of any suitably flexible, easily molded material such as silicone rubber or thermoplastic elastomer. Sheet 20 is attached or bonded to substrate 22 by any suitable technique such as adhesive, heat fusion or mechanical crimping. Substrate 22 preferably is made of a material that is rigid with respect to sheet 20, such as a rigid thermoplastic, and may be made by any suitable method, such as machining or injection molding. Cassette 18 is generally formed separately from console 200 and held in operative association with console 200 by fluid connections and latching mechanisms well-known in the art.

Additionally, as best shown in FIG. 2, cassette 18 has an outlet flow channel 32 extending from and in fluid communication with outlet port 28. More specifically the outlet flow channel 32 has a liquid trap 34 that is positioned directly adjacent to and in fluid communication with outlet port 28. For example, liquid trap 34 can be similar to a p-trap or any other structure designed to trap liquid adjacent outlet port 28. Liquid trap 34 is oriented such that the trap extends substantially upward initially in the direction of arrow A from outlet port 28. In that regard, arrow A points in a direction that substantially opposes the direction of gravity. The liquid trap 34 represents a liquid reservoir that traps liquid adjacent the outlet port 28. In fact, because the liquid trap 34 enables the trapping of liquid adjacent outlet port 28 any gas that otherwise would be present near outlet port 28 is removed during the cassette priming process. Liquid trap 34 preferably is made of a material that is rigid, such as a rigid thermoplastic, and may be made by any suitable method, such as machining or injection molding.

As will be described in greater detail below, liquid trap 34 traps a volume of liquid within the outlet flow channel 32 that is greater than or equal to the volume of expansion for channel 26 to prevent gas from being able to be backfilled (i.e. sucked) into channel 26 during use of peristaltic pump 10. For example, liquid trap 34 can have a volume of about 0.1 cc for trapping liquid while the volume of expansion for channel 26 is equal to or less than about 0.1 cc. However, in other embodiments liquid trap 34 can have a volume that is greater than or less than about 0.1 cc as long at the volume of liquid trap 34 is greater than or equal to the volume of expansion for channel 26.

In use, cassette 18 is held in close proximity to roller head 14 so that rollers 16 compress channel 26 against substrate 22 as roller head 14 rotates. The longitudinal axes of the rollers are arranged so that rollers 16 contact with channel 26 is generally parallel with the plane of channel 26. Such an arrangement eliminates the need to loop a length of flexible tubing over the pump roller head and thus simplifies the loading of pump channel 26 against pump roller head 14. Rollers 16 may be tapered along their axial length to accommodate the difference in path length traveled by the inner and outer sections of rollers 16 as roller head 14 rotates. As described in U.S. Publication No. 2007/0207041 titled “Method of Operating a Peristaltic Pump,” which is hereby incorporated by reference in its entirety, unwanted pressure pulsations can be minimized by providing channel transition regions having internal cross-sections that taper from zero to the full cross-section of channel 26. These regions minimize the abrupt change in displaced volume as rollers 16 transition on or off of channel 26.

Moreover, during use, the rollers 16 of roller head 14 compress sheet 20 against substrate 22 thereby compressing channel 26 to drain fluid through pump 10. The compression of rollers 16 against sheet 20 along channel 26 between the inlet port 30 and the outlet port 28 provides an isolation function when the inlet port 30 is subjected to a vacuum condition. The quality of this isolation function (i.e. roller pinch point) depends on the loading force and the alignment of the roller 16 with respect to sheet 20 and substrate 22. Ideally this compression (or pinching by the roller) would completely seal the pump preventing any leakage between the upstream and downstream portions of the pump. However, this is not always the case and small leaks may occur.

In the event of a leak in the seal, gas can easily escape upstream through the roller pinch point when the inlet port of a drain pump is exposed to a vacuum condition. In such a case, once the gas is in an upstream chamber of the pump the gas will expand when exposed to the vacuum condition and thereby affect the operability of the pump. Because of the adverse effect that gas can have upstream on a pump it is preferable to have liquid downstream of a leak instead of gas. In that regard, liquid will leak more slowly than gas due to its higher viscosity and once in the upstream chamber liquid will not expand as a gas would when exposed to a low pressure condition. Therefore, by ensuring liquid is present downstream of the seal formed by the rollers 16, the harmful effect of a gas leak is prevented and the isolation function of the roller is less sensitive to the loading force and misalignment of the rollers 16 with respect to the sheet 20 and substrate 22 along channel 26.

Here, liquid trap 34 prevents gas from escaping upstream within pump 10. For example, when the inlet port 30 of pump 10 is exposed to a vacuum condition, the channel 26 is contracted before being pinched off by at least two rollers 16 of roller head 14. Subsequently, as identified in FIG. 1, when one of the rollers 16 is rotated beyond the outlet port 28 a portion of channel 26, identified as expanded portion E, or expansion volume E, that was previously contracted will lose its vacuum and expand from the outlet port 28 to the pinch point being applied by the subsequent roller 16. Normally, this expansion of channel 26 would cause a volume of liquid and/or gas located near outlet port 28 to be backfilled (i.e. sucked) into channel 26 in a proportion equal to the volume of expansion undergone by channel 26. If any gas were present in the outlet flow channel 32 near the outlet port 28, this gas may be backfilled into the newly expanded portion E of channel 26.

However, as discussed above, during priming of pump 10 liquid trap 34 traps a volume of liquid that is greater than or equal to the volume of expanded portion E of channel 26 that occurs when one of the rollers 16 is rotated beyond the outlet port 28 causing the previously contracted channel 26 to lose its vacuum and expand. Because liquid trap 34 traps only liquid adjacent outlet port 28 at a volume that is greater than or equal to the volume of expanded portion E there will only be liquid present in the outlet flow channel 32 adjacent outlet port 28 that is capable of being backfilled into the expanded portion E of channel 26. Therefore, in the event of a leak in the seal formed by the subsequent roller 16 that has yet to roll pass the outlet port 28, only liquid will be present within expanded portion E of channel 26 to leak through the faulty seal thereby avoiding the adverse effects associated with a gas leakage between the upstream and downstream portions of the pump. Thus, the isolation function of rollers 16 can be additionally influenced by the degree of how well the outlet flow channel 32 is primed to insure that liquid trap 34 traps a volume of liquid that is greater than or equal to the volume of expanded portion E of channel 26.

It should be noted that in an alternative embodiment liquid trap 34 can have a volume capable of trapping a liquid that is greater than the entire length of channel 26. In other words, liquid trap 34 can have a volume capable of trapping a liquid that is greater than expanded portion E by having a volume that is greater than or equal to the entire expandable volume of channel 26.

Furthermore, in another alternative embodiment liquid trap 34 can have a volume capable of trapping a liquid that is less than expanded portion E. In such an embodiment, liquid trap 34 insures there will be liquid present in the outlet flow channel 32 adjacent outlet port 28 that is capable of being backfilled into the expanded portion E of channel 26. Therefore, in the event of a leak in the seal formed by the subsequent roller 16 that has yet to roll pass the outlet port 28, liquid will be present within expanded portion E of channel 26 to leak through the faulty seal thereby diminishing and/or avoiding the adverse effects associated with a gas leakage between the upstream and downstream portions of the pump. Thus, the isolation function of rollers 16 can be influenced by the degree of how well the outlet flow channel 32 is primed to insure that liquid trap 34 traps a volume of liquid adjacent outlet port 28 that is capable of being backfilled into the expanded portion E of channel 26.

FIG. 5 is a schematical view of cassette 18 of peristaltic pump 10 positioned within surgical console 200. As shown, cassette 18 is connected to a reservoir 100 and a drain bag 110. Reservoir 100 is connected to surgical handpiece 114 through aspiration line 112 and connected to peristaltic pump 10 through pump inlet line 116. Vacuum line 118 enters reservoir 100 near top 120 of reservoir 100 and is connected to a source of vacuum, such as a venturi pump (not shown), contained within console 200. When vacuum is applied to vacuum line 118, such vacuum is communicated to reservoir 100; thereby drawing fluid through handpiece 114 via aspiration line 112.

When reservoir 100 becomes sufficiently filled with fluid, peristaltic pump 10 is activated to draw fluid off of bottom 122 of reservoir 100 through pump inlet line 116 and inlet port 30 and discharges the excess fluid into drain bag 110 through outlet port 28 and pump discharge line 124. Moreover, as discussed above, outlet port 28 has a liquid trap 34 that is positioned directly adjacent to and in fluid communication with outlet port 28. In that regard, liquid trap 34 traps a volume of liquid that is greater than or equal to the volume of expansion for channel 26 that occurs when one of the rollers 16 is rotated beyond the outlet port 28 causing the previously contracted channel 26 to lose its vacuum and expand. Therefore, in the event of a leak in the seal formed by a subsequent roller 16 that has yet to roll pass the outlet port 28, only liquid will be present within expanded portion E of channel 26 to leak through the faulty seal thereby avoiding the adverse effects associated with a gas leakage between the upstream and downstream portions of the pump.

In an alternative embodiment, instead of a vacuum being applied to vacuum line 118, the aspiration line may be direct connected to the inlet port 30. In this case, the peristaltic pump itself is functioning as a vacuum source.

While the present invention has been illustrated by the above description of embodiments, and while the embodiments have been described in some detail, it is not the intention of the applicant to restrict or in any way limit the scope of the invention to such detail. Additional advantages and modifications will readily appear to those skilled in the art. Therefore, the invention in its broader aspects is not limited to the specific details, representative apparatus and methods, and illustrative examples shown and described. Accordingly, departures may be made from such details without departing from the spirit or scope of the applicant's general or inventive concept.

What is claimed is:

1. A cassette for use with a peristaltic pump, the cassette comprising:
a body having an exterior;
a sheet attached to the body forming at least one fluid channel, the fluid channel being generally arcuate and projecting outwardly from the exterior of the body, the fluid channel having at least one outlet port for receiving a liquid; and
a liquid trap in fluid communication with and extending at least in part in an upward direction from the at least one outlet port.

2. The cassette of claim 1, wherein the upward direction is a direction that substantially opposes the direction of gravity.

3. The cassette of claim 1, wherein the exterior of the body has opposing side surfaces such that the sheet extends outwardly from one of the opposing side surfaces and the liquid trap extends outwardly from the other of the opposing side surfaces.

4. The cassette of claim 1, wherein the liquid trap is a liquid reservoir that traps liquid adjacent the at least one outlet port to prevent gas from accessing the fluid channel through the at least one outlet port during use of the cassette.

5. The cassette of claim 1, wherein the fluid channel has an expansion volume and the liquid trap traps a volume of liquid that is greater than or equal to the expansion volume of the fluid channel.

6. The cassette of claim 1, wherein the at least one outlet port is configured for draining fluids.

7. The cassette of claim 1, wherein the liquid trap is positioned directly adjacent to the at least one outlet port and is part of an outlet flow channel for draining fluids.

8. A cassette for use with a peristaltic pump, the cassette comprising:
a body having an exterior;
a sheet attached to the body forming at least one molded fluid channel, the fluid channel being generally arcuate and projecting outwardly from the exterior of the body to define an expansion volume, the fluid channel having at least one outlet port for receiving a liquid; and
a liquid trap in fluid communication with the at least one outlet port, the liquid trap defining a volume that is greater than or equal to the expansion volume of the fluid channel.

9. The cassette of claim 8, wherein the liquid trap extends at least in part in an upward direction from the at least one outlet port.

10. The cassette of claim 9, wherein the upward direction is a direction that substantially opposes the direction of gravity.

11. The cassette of claim 9, wherein the liquid trap is positioned directly adjacent to the at least one outlet port.

12. The cassette of claim 9, wherein the liquid trap is a liquid reservoir that traps liquid adjacent the at least one outlet port to prevent gas from accessing the fluid channel through the at least one outlet port during use of the cassette.

13. The cassette of claim 8, wherein the exterior of the body has opposing side surfaces such that the sheet extends outwardly from one of the opposing side surfaces and the liquid trap extends outwardly from the other of the opposing side surfaces.

14. A method for operating a peristaltic pump having a roller head, the method comprising:
providing a surgical cassette comprising:
a body having an exterior;
a sheet attached to the body forming at least one fluid channel, the fluid channel having an inlet port and an outlet port; and
a liquid trap in fluid communication with and extending at least in part in an upward direction from the outlet port;
positioning the cassette with respect to the roller head of the peristaltic pump;
rotating the roller head such that at least one roller contacts the sheet to cause fluid flow through the at least one fluid channel; and
trapping liquid with the liquid trap adjacent the outlet port.

15. The method of claim 14, further comprising exposing the inlet port to a vacuum condition thereby contracting the sheet in the direction of the exterior of the body to contract the fluid channel.

16. The method of claim 15, wherein rotating the roller head such that the at least one roller contacts the sheet includes rotating the at least one roller past the outlet port such that the sheet expands outwardly from the exterior of the body to expand the fluid channel.

17. The method of claim 16, further comprising filling the fluid channel with only the liquid trapped adjacent the outlet port when the vacuum is lost and the sheet expands outwardly from the exterior of the body to expand the fluid channel.

18. The method of claim 17, wherein the trapped liquid adjacent the outlet port has a volume that is equal to or greater than an expansion volume of the fluid channel when the sheet expands outwardly from the exterior of the body to expand the fluid channel.

19. The method of claim 14, wherein trapping liquid with the liquid trap adjacent the outlet port occurs during priming of the peristaltic pump

20. The method of claim 14, wherein the upward direction is a direction that substantially opposes the direction of gravity.
